(19) United States
(12) Patent Application Publication
(10) Pub. No.: US 2002/0044888 A1
(43) Pub. Date: Apr. 18, 2002

(54) Cleaning of a workpiece by pulsatile pressure

(76) Inventors: John W. Morris, Beachwood, NJ (US); Lawrence A. Shimp, Morganville, NJ (US); Nelson L. Scarborough, Andover, MA (US); James L. Russell, Little Silver, NJ (US)

Correspondence Address: Peter G. Dilworth, Dilworth & Barrese, 333 Earle Ovington Blvd., Uniondale, NY, 11553, US

(21) Appl. No.: 09/780,216

(22) Filed: Feb. 9, 2001

Related U.S. Application Data
(60) Provisional application No. 60/181,205, filed on Feb. 9, 2000.

(51) Int. Cl. A61L 2/16

(52) U.S. Cl. 422/33; 422/295

(57) ABSTRACT

There is provided a pulsatile flow pressure system for treating tissue, such as, for example, bone tissue. This system generally includes one, and preferably two, pressure chambers configured to receive the natural ends of intact bone. Alternate positive and negative pressures are enacted on the bone ends to force fluid into the bone and substantially out through an exterior surface thereof. Alternatively, both ends of an intact bone can be positioned in separate pressure chambers and simultaneous positive or negative pressures applied to the bone ends to force fluid through the bone tissue.

### CROSS-REFERENCE TO RELATED APPLICATIONS

The present disclosure claims priority to U.S. Provisional Application Serial No. 60/181,205, filed Feb. 9, 2000, and entitled, “Cleaning of a Workpiece by Pulsatile Pressure”.

### BACKGROUND

1. Technical Field

This invention relates to an apparatus and method for treating, e.g., cleaning, the interior of a fluid permeable workpiece by establishing a cyclic or pulsatile flow of fluid using pressure differentials. More specifically, the present invention provides a pressure flow system for accessing the internal porous matrix of bone that is intended for implantation with one or more treatment fluids to variously clean, defat, sterilize, virally inactivate, disinfect, and/or demineralize the bone, to facilitate the visualization of its structure or to impregnate the bone with one or more pharmacological agents (antibiotics, bone growth factors, etc.) so the bone can act as a drug delivery system.

2. Background of Related Art

The preparation of bone tissue for subsequent implantation involves one or more aseptic cleaning procedures that are intended to minimize the risk of transferring potential harmful disease organisms to tissue recipients and to reduce transplant-related antigenicity. Known bone cleaning procedures are not always completely and/or consistently effective. Accordingly, there is a risk that in employing some of the known bone cleaning procedures, harmful microorganisms and/or antigenic material may continue to reside in bone.

U.S. Pat. No. 5,333,626 discloses a method for preparing bone for transplantation in which the bone is first contacted with a decontaminating agent and subsequently contacted with detergent under high pressure washing conditions at elevated temperatures. U.S. Pat. Nos. 5,513,662 and 5,556,379 disclose the application of a less than ambient pressure, i.e., vacuum to facilitate displacement of removable material from the internal matrix of bone as a method of preparing the bone for transplantation. Although the above-described methods have realized some success, difficulties have been encountered in uniformly penetrating deep into the bone matrix where infectious agents and/or immunogenic macro-molecules may be present.

An exemplary system for forcing fluid through a workpiece in single direction is disclosed in U.S. Pat. No. 5,846,484, issued Dec. 8, 1998, and entitled “Pressure Flow System and Method for Treating a Fluid Permeable Workpiece Such as Bone”, the entire disclosure of which is incorporated by reference herein. However, this system may not be optimal for treatment of intact structures, such as, intact bone.

Accordingly, a need exists for an improved apparatus and process for cleaning and decontaminating an intact entire bone structure or a portion of a bone by cyclical or pulsatile pressure and which minimizes exposure of a transplant recipient to potentially harmful diseases and transplant related antigenicity. The disclosed system further provides a method to treat bone with agents that can result in improved performance characteristics to act as a means to deliver one or more bioactive agents to a body in which the bone is implanted, to allow for staining of bone for diagnostic or research purposes or to study the fluid flow characteristics of bone and its microvasculature.

### SUMMARY

The present disclosure relates to a chamber system in which both positive and negative pressures are utilized to force fluid through a workpiece. Alternating positive and negative cycles are used to enact pressure gradients between the interior and exterior of a porous workpiece thereby forcing the fluid through the interior of the workpiece and out the exterior surface. Uses of this system are to drive the decontaminating solutions into the workpiece and/or remove the removable elements from the interior of the workpiece. Preferably, the positive pressures used range from approximately 5 to 100 psi while negative pressure applied is approximately from 0 to about 14 psi. A preferred use for the disclosed system is for treating a bone tissue.

In one embodiment, a single pressure chamber is employed to provide alternate positive and negative pressures on an end of an intact bone thereby enacting a cyclic or pulsatile flow of treatment fluid through the bone. As used herein, “intact bone”, shall be understood to mean a complete natural bone that has not been split, sectioned or otherwise cut at a point between the natural ends thereof.

In a second embodiment, two separate pressure chambers are employed to provide alternate positive and negative pressures on opposing ends of a bone thereby enacting a cyclic or pulsatile flow. Alternately, both chambers may be pressurized to substantially the same positive and negative pressures in cycles. The present system is also suitable for treatment of small and irregular pieces, and can be advantageously used to treat intact bones which are later cut to final product sizes.

In the disclosed embodiments, the fluid permeable workpiece is a bone or a section thereof and the fluid is a cleaning or disinfecting fluid which is forced to flow through the vasculature and porous structure of the bone to effect the removal of blood, bone marrow and/or other non-bone constituent(s) from the bone, and/or sterilize and virally inactivate the bone. Alternately, the pressure flow system can be employed to demineralize bone, to stain the microvasculature of bone to improve its visualization or to introduce bioactive agents.

The expression “fluid permeable workpiece” as used herein shall be understood to include any article, device, material, or the like, which permits the passage of a fluid under pressure therethrough. The term “fluid” includes all liquid and gaseous treatment substances, and their mixtures, that are flowable under the conditions of operation of the pressure flow system.

### BRIEF DESCRIPTION OF THE DRAWINGS

Various embodiments are described herein with reference to the drawings, wherein:

FIG. 1 is a perspective view of a first embodiment of a pulsatile pressure flow system having a single pressure chamber; and

FIG. 2 is a perspective view of a second embodiment of a pulsatile pressure flow system having dual pressure chambers.

### DETAILED DESCRIPTION OF PREFERRED EMBODIMENTS

Preferred embodiments of the presently disclosed pressure flow system will now be described in detail with reference to the drawings in which like reference numerals designate identical or corresponding elements in each of the several views.

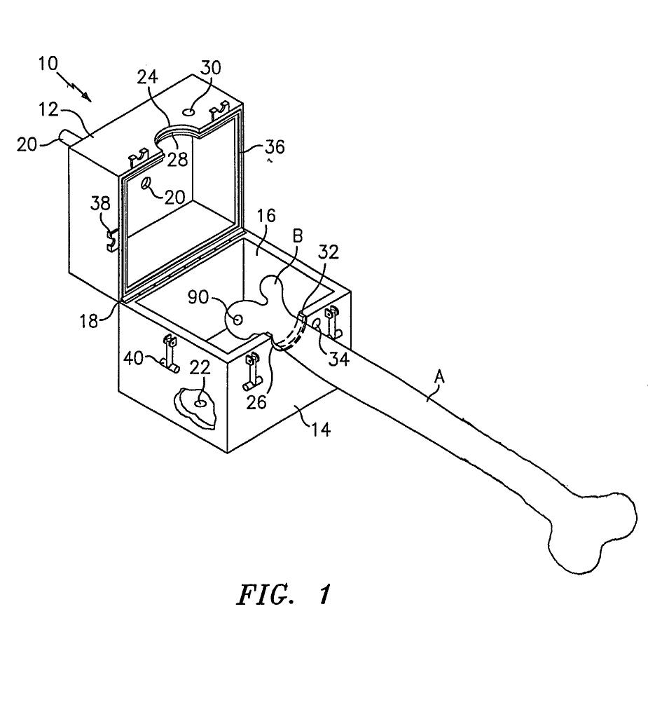

Referring to FIG. 1, the disclosed system generally includes a first chamber 10 having an upper chamber section 12 and a lower chamber section 14. Upper chamber section 12 and lower chamber section 14 are combined to form an interior chamber 16 for receipt of pressurized fluids and partial portions of a workpiece. Preferably, upper chamber section 12 and lower chamber section 14 are connected together by a hinge 18 extending along edges thereof. First chamber 10 is provided with a fluid inlet port 20 in an upper surface of chamber 10 and a fluid outlet port 22, preferably, in the lower surface thereof. These ports connect to pressure and/or vacuum sources. Inlet port 20 and outlet port 22 preferably contain check valves. Alternate locations of inlet and outlet ports 20, 22, respectively, are also contemplated.

In order to receive a portion of a workpiece, such as, for example, a section of bone tissue, upper chamber section 12 is provided with an upper cutout 24 and lower chamber section 14 is provided with a lower cutout 26. An upper inflatable seal 28 is formed within upper cutout 24 and is connected to an air port 30. Similarly, a lower inflatable seal 32 is provided within lower cutout 26 and is connected to an air port 34. By inflating upper and lower inflatable seals 28 and 32, respectively, a fluid tight seal may be obtained around the irregular surfaces of the bone positioned therein. A gasket 36 may be provided along an edge of upper chamber section 12 and/or lower chamber section 14 to provide a fluid tight seal therebetween. Preferably, upper chamber section 12 and lower chamber section 14 are secured together by upper locks 38 which engage lower thumb screws 40. Alternate and/or known methods of sealingly securing upper chamber section 12 to lower chamber section 14 are also within the scope of the present disclosure.

While the disclosed pressure chamber is illustrated as chamber 10, as shown, other known pressure chambers and pressure/fluid sources, including various controls, valves, sensors etc., such as those disclosed in U.S. Pat. No. 5,846,484 can be advantageously used to supply alternate positive and negative pressures and treatment fluids to bone ends.

Referring again to FIG. 1, the disclosed pressure flow system is particularly suited for treating bones, including cadaveric long bones, ilium, ribs, etc., intended for surgical implantation. In a preferred embodiment of the pressure flow system for treating bone, a fluid subsystem is connected to inlet port 20 and includes a surface active agent of the anionic, cationic, amphoteric and/or non-ionic variety. Preferably, the surface active agent is a non-ionic octyphenoxy polyethoxy ethanol 26 suitable for removing protein and lipids from bone, such as Triton′X-100™ (Rohm and Haas Co.), although other surface active agents can also be used. For example, a fluid pump, which is preferably a peristaltic pump, although other kinds of pumps can be used, supplies the surface active agent to a manifold through an electrically controlled valve. A flow controller is operatively connected to a flow transducer to control the speed of the pump and thereby control the concentration of surface active agent within the manifold.

Cationic surfactants which can be employed include quaternary amino or nitrogen compounds; quaternary ammonium salts such as benzalkonium chloride, alkyltrymethylammonium salts, and alkylpyridinium salts; aliphatic mono-di, and polyamines; rosin-derived amines; amine oxides, such as polyoxyethylene alkyl and alicyclic amines. N.N.N.N. tetrakis-substituted ethylene diamines, amidelinked amines, preferably those prepared by the condensation of a carboxylic acid with a di- or polyamine, and sodium tauro 24, 25 dihydrofusidate.

Anionic surfactants which can be employed include sulfates such as alkyl sulfates (for example, sodium dodecyl sulfate), sulfated fats and oils, sulfated oleic acid, sulfated alkanollamides, sulfated esters, and alcohol sulfates; sulfonates such as alkylaryl sulfonates, olefin sulfonates, ethoxylated alcohol sulfates, and sulfonates of ethoxylated alkyl phenols; sulfates of fatty esters; sulfates and sulfonates of alkyl phenols; lignosulfonates; sulfonates of condensed naphthalenes; sulfonates of naphthalene, dialkyl sulfosuccinates, preferably sodium derivatives; sodium derivatives of sulfo-succinates, such as the disodium ethoxylated nonyl phenol half ester of sulfosuccinic acid, the disodium ethoxylated alcohol (C₁₀-C₁₁), half-ester of sulfosuccinic acids, etc., petroleum sulfonates, such as alkali salts of petroleum sulfonates; for example, sodium petroleum sulfonate (Aco 632); phosphate esters, such as alkali phosphate esters, and a potassium salt of phosphate ester (Triton H66); sulfonated alkyl esters (for example, Triton GR7); carboxylates, such as those of the formula (RCOO)−(M)+wherein R is an alkyl group having from 9-21 carbon atoms, and M is a metal or an amine; and sodium polymeric carboxylic acid (Tamol 731) and the like.

Non-ionic surfactants which can be employed include polyoxyethylenes; ethoxylated alkyl phenols, ethoxylated aliphatic alcohols; carboxylic acid esters, such as glycerol esters, polyethylene glycol esters, and polyoxyethylene fatty acid esters; anhydrosorbitol esters and ethoxylated anhydrosorbitol esters; glycol esters of fatty acids; ethoxylated natural fats, oils, and waxes; carboxylic amides, such as diethanolamine condensates, and monoalkanolamine condensates; polyoxyethylene fatty acid abides; polyalkylene oxide block copolymers, preferably polyethylene and polypropylene oxide block copolymers; and polysiloxane-polyoxylkylene copolymers; 1-dodecylazacyeloheptan-one polyethylene glycol monolaurate; and Macrochem's SEPA non-ionic surfactant.

Preferred non-ionic surfactants are ethylene oxide condensation products (polyoxyethylene) containing more than two, and preferably at least five, ethylene oxide groups with at least one end group thereof being terminated by reaction with either an alcohol alkylphenol or a long chain fatty acid. A particularly preferred non-ionic surfactant is an octylphenoxy polyethoxyethanol surfactant known as Triton X-100.

Amphoteric surfactants include N-coco-3 aminopropionic acid and its sodium salt; disodium salts of N-tallow-3-iminodipropionate and N-lauryl-3-iminodipropionate; N-carboxymethyl-N cocoalkyl-N-dimethylammonium hydroxide; N-carboxymethyl-N-dimethyl-N-(9 octadecenyl) ammonium hydroxide; (1-carboxy heptadecyl) trimethylammonium hydroxide; (1-carboxyundecyl) trimethylammonium hydroxide; sodium salts of N-cocoamidoethyl-N-hydroxyethylglycine and N-hydroxyethyl-N-stearamido-glycine; sodium of salts of N-hydroxyethyl-N-lauramido-B-alamine and N-cocoamido-N-hydroxyethyl-B-alanine; sodium salts of mixed alicyclic amines, ethoxylated and sulfated sodium salts or free acids of 2-alkly-1-carboxymethyl-1-hydroxyethyl-2-imidazol hydroxide; the disodium salt of 1.1-bis (carboxymethyl)-2-undecyl-2-imidazolinium hydroxide; and the sodium salt of a propoxylated and sulfated oleic acid-ethylenediamine condensate.

Another fluid subsystem can include a source of acid which is supplied to the manifold via inlet and outlet valves by the pump. A pH controller is operatively associated with a pH transducer mounted on the manifold to control the speed of the acid pump and maintain the treatment solution in the manifold at a constant pH. Preferably, the pH controller also is operatively associated with a flow transducer to maintain a more precise control over acid delivery. Acids which can be employed in this operation include inorganic acids such as hydrochloric acid and organic acids such as peracetic acid. After acid treatment, the bone is rinsed with sterile clean process water, buffered with a buffering agent to a final predetermined pH and then finally rinsed with clean process water to remove residual amounts of acid and buffering agent.

In yet another fluid subsystem, the subsystem can include a source of ethanol which is supplied to the manifold through electrically controlled valves by the pump. A controller is operatively connected to a pressure transducer mounted on chamber 10 to control the speed of the ethanol pump. The overall sequence and timing of the operations of various fluid subsystems are controlled by operator input and a master controller, which is operatively associated with the flow transducer to coordinate operation of each of these subsystems. Each subsystem can be operated alone or in combination with any of the other subsystems to treat bone.

Other fluids can also be provided in the disclosed system in addition to or in combination with those already mentioned. For example, a preferred defatting/disinfectant solution is an aqueous solution of ethanol and non-ionic surfactant, the ethanol being an effective solvent for lipids and the water being an effective hydrophilic carrier to enable the solution to penetrate more deeply into the bone. The aqueous ethanol solution also disinfects the bone by killing vegetative micro organisms and viruses. For example, the non-ionic surfactant destroys the lipid toga viruses such as HIV and HBV. Ordinarily at least about 10% to 40% water (i.e., about 60% to 90% defatting agent such as alcohol) should be present in the defatting, disinfecting solution to produce optimal lipid removal and disinfection within the shortest period of time. The preferred concentration range of the defatting solution is about 60% to 85% alcohol and most preferably about 70% alcohol.

Medically/surgically useful substances which can be supplied by pressure flow system 10, in addition to those set forth above include, e.g., collagen, insoluble collagen derivatives, bydroxypatite, etc., and soluble solids and/or liquids dissolved therein, e.g., antiviricides, particularly those effective against HIV and hepatitis; antimicrobials and/or antibiotics such as erythromycin, bacitracin, neomycin, penicillin, polymyxin B, tetracyclines, viomycin, chloromycetin and streptomycins, cefazolin, ampicillin, azactam, tobramycin, clindamycin and gentamicin, etc.; amino acids, magainins, peptides, vitamins, inorganic elements, inorganic compounds, co-factors for protein synthesis; hormones; endocrine tissue or tissue fragments; synthesizers, enzymes such as collagenase, peptidases, ozidases, etc.; polymer cell scaffolds with paraenchymal cells, surface cell antigen eliminators; angiogenic drugs and polymeric carriers containing such drugs; collagen lattices; biocompatible surface active agents; antigenic agents; cytoskeletal agents; cartilage fragments; living cells such as chondrocytes, bone marrow cells, mesenchymal stem cells, natural extracts, tissue transplants, bioadhesives, bone morphogenic proteins (BMPs), transforming growth factor (TGF-beta), insulin like growth factor (IGF-1); platelet derived growth factor (PDGF), fibroblast growth factors (FGFF), vascular endothelial growth factor (VEGF), angiogenic agents, bone promoters, cytokines, interleukins, genetic material, genes encoding bone promoting action, cells containing genes encoding bone promoting action; growth hormones such as somatotropin; bone digestors; antitumor agents; fibronectin; cellular attractants and attachment agents, immumunosuppressants; permeation enhancers, e.g., fatty acid esters such as laureate, myristate and stearate monoesters of polyethylene glycol, enamine derivatives, surface active agents, alphaketo aldehydes, etc.; nucleic acids, and, biorodable, polymers such as those disclosed in U.S. Pat. Nos. 4,764,364 and 4,765,973 and European Patent Application 168,277. The amounts of such optionally added substances can vary widely with optimum levels being readily determined in a specific case by routine experimentation.

Bone is composed of two types of tissue, cortical tissue and cancellous tissue. The periosteal portion of bone is formed of cortical tissue which has a porous structure with a large quantity of solid matter. The endosteal portion of bone is formed of cancerous tissue which has a sponge-like appearance and also includes a porous structure having smaller amounts of solid matter. The relative quantity of each kind of tissue varies in different bones and within different parts of the same bone to meet the requisite strength requirements of the bone. The interior of long bones includes a central cavity called the medullary canal.

During treatment of bone using the disclosed pressure flow system, fluid from within chamber is forced to flow from the periosteal portion of bone to the endosteal portion of bone to a position within a portion of bone located outside chamber 10. The fluid is then forced to flow from the endosteal portion of the bone to the periosteal portion of the bone to exit bone outside chamber 10. It has been discovered that forcing the treatment fluid to flow along the natural circulatory path of bone, i.e., from the endosteal portion of bone to the periosteal portion of bone, results in more uniform and deeper fluid penetration into the bone matrix, especially in the cortical tissue of bone.

In use, a bone section such as long bone A having an end B is positioned within lower cutout 26 and upper chamber section 12 is closed towards lower chamber section 14. Upper chamber sections 12 and lower chamber sections 14 are thereafter secured by locking lower thumb screws 40 with upper locks 38. Additionally, upper and lower inflatable seals 28 and 32 are inflated to provide a fluid tight seal around the irregular surface of bone A positioned therein. Optionally, holes 90 can be drilled in one or both ends of the bone to facilitate fluid flow.

Once bone end B has been positioned within interior 16 of first chamber 10, first chamber 10 is pressurized with a fluid, for example, a cleaning fluid such as that disclosed in U.S. Pat. No. 5,846,484 referred to herein above. The fluid is forced through the check valve in fluid inlet port 20 to a pressure between about 5 psi to 1500 psi. Preferably first chamber is pressurized to a pressure of approximately 5 to 100 psi. The fluid is thereafter forced through the porous end of bone B to flush and/or treat the interior of bone A. A cut or hole 90 may be formed in the end of the bone to facilitate fluid flow. At the end of the first treatment cycle, fluid ceases to be forced through fluid inlet port 20 and a check valve in fluid outlet port 22 opens to allow drainage of the fluid there through. Next, a second cycle applying a negative pressure of about 0 to 14.7 psi is generated within interior 16 of first chamber 10 to thereby draw out contaminants from bone end B. This process may be repeated through several cycles utilizing cleaning and/or treatment fluids to treat a section of bone A.

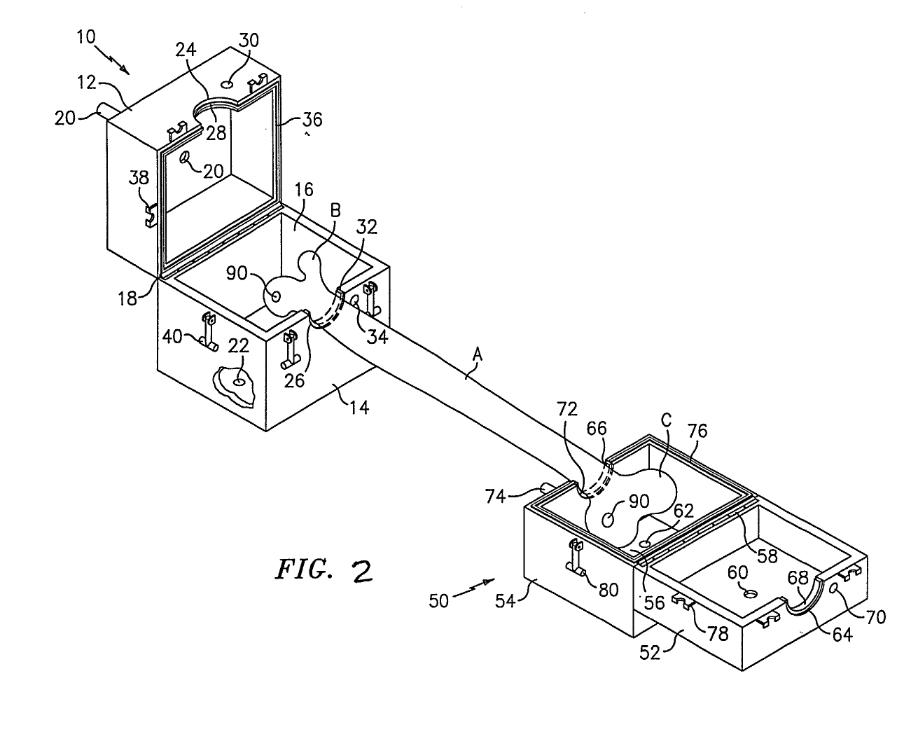

Referring now to FIG. 2, in an alternative embodiment, a second chamber 50, similar to chamber 10, is provided with an upper chamber section 52 and a lower chamber section 54 which together define an interior chamber 56. Upper chamber section 52 and lower chamber section 54 are also connected by a hinge 58 and include a fluid inlet port 60 and a fluid outlet port 62. Upper chamber section 52 is provided with an upper cutout 64 and lower chamber section 54 is provided with a lower chamber cutout section 66. Upper inflatable seals 68 having an air port 70 and lower inflatable seal 72 have an air port 74 are provided in upper and lower chamber sections 52 and 54, respectively. Additionally, second chamber 50 may be provided with a gasket 36 and upper lockdowns 78 and lower thumbscrews 80 to provide a sealed system.

In use, a bone section, such as bone section A, having a first end B and a second end C, is positioned with first end in first chamber 10 and second end in second chamber 50 in the manner described herein above. Optionally, cuts or holes 90 can be drilled or otherwise formed in one or both ends of the bone to facilitate fluid flow. Alternating pressure cycles between first chamber 10 and second chamber 50 are provided to achieve a pulsatile type flow through the bone section in order to cleanse, decontaminate and/or treat the bone using the pressure ranges described above. The difference in magnitude of the positive and negative pressures results in substantially all of the fluid being forced out the exterior surface of the bone. For example, a high pressure may be provided in a first chamber 10 whereas a negative pressure may be provided in second chamber 20 to pass fluid through bone section A and substantially out the exterior surface thereof. Subsequent to that, the pressures are reversed resulting in a negative pressure in first chamber 10 and a higher positive pressure in second chamber 50 to thereby drive the fluids and/or remove materials back through the bone section. Alternatively, first and second chambers 10 and 50, respectively, may be simultaneously pressurized to substantially the same positive and negative pressures to achieve the cyclic flow process.

As discussed above, the disclosed pressure low system can be used to perform treatment procedures besides cleaning. For example, the system can be used to effect demineralizaton of bone by forcing an acid or other demineralizing agent through the bone. This can be accomplished by activating a water pump and an acid pump to supply an acidic solution to pressure chamber 10. After a specified duration, the acid pump can be deactivated and the water pump operated alone to flush the acid from the bone. Where bone has any significant cross-sectional dimension, current methods of demineralization (e.g., acid bath) result in demineralization progressing by a solvent front process. This results in bone which retains some amount of mineral matter that increases towards its center. By contrast, the pressure flow system described herein provides a demineralized bone possessing a more uniform demineralization profile than such current methods, essentially increasing the porosity by demineralizing the vascular channels throughout the bone.

This system is intended to be used to develop allograft forms that can provide structural support, but that have enhanced ability to be remodeled into host bone due to improved vascular access through the demineralized microporous structure of bone. This process will allow for development of osteoinductive weight bearing allografts.

It will be understood that various modification can be made to the embodiments disclosed herein. For example, various combinations of the cyclic and/or simultaneous pressure flow methods may be combined or used together when processing tissue. The flow system need not be used for cleaning or demineralization but can instead be used as a histology tool. Because the pressure flow system described above has proved to be effective at penetrating mineralized tissue, the system can be used to inject a dye or any other suitable contrasting agent, e.g., a methylene blue dye, into the smallest recesses of bone including the osteocytic lacunae to improve visualization of these minute structures and to better enable the study of the microvasculature of bone. The chamber can also be used to study fluid flow mechanics through bone microvasculature. It can also be used to impregnate bone with pharmacological agents (antibiotics, bone growth factors, etc.) so the bone can act as a delivery system. Further, more than one pressure chamber can be used concurrently to treat one or several workpieces. For example, a whole bone can be treated by securing one end of the bone within a first pressure chamber and the other end of the bone within a second pressure chamber. In yet another example, a pressure chamber can include more than one opening having a seal positioned in each opening to facilitate simultaneous treatment of several workpieces employing a single chamber. Therefore, the above description should not be construed as limiting but merely as exemplifications of preferred embodiments. Those skilled in the art will envision other modifications within the scope and spirit of the claims appended thereto.

1. A pressure flow system for treating the interior of permeable bone with a fluid which comprises:
a) a first fluid pressure chamber having a fluid inlet port and an opening formed through a wall of the chamber sized to allow passage of at least a portion of the bone there through;
b) a seal positioned about the opening conformable to the surface of the bone and capable of fluid-tight engagement therewith;
c) a first source of pressurized fluid having a pressure greater than atmospheric pressure;
d) a second fluid pressure chamber having a fluid inlet port and an opening formed through a wall of the chamber sized to allow passage of at least a portion of the bone there through;
e) a seal positioned about the opening conformable to the surface of the bone and capable of fluid-tight engagement therewith; and
f) a second source of pressurized fluid having a pressure less than atmospheric pressure, wherein when the first fluid pressure chamber is pressurized to a pressure greater than atmospheric pressure, fluid from the first fluid pressure chamber flows from the first fluid pressure chamber through a first end of the interior of the bone and exits the bone outside the first fluid pressure chamber; and wherein when the second fluid pressure chamber is pressurized to a pressure less than atmospheric pressure, fluid from within the bone flows from the bone through the interior of the bone and exits the bone inside the second fluid pressure chamber.

2. The pressure flow system as recited in claim 1 wherein the first fluid pressure chamber is pressurized to a pressure of approximately 5 psi to 1500 psi.

3. The pressure flow system as recited in claim 2 wherein the first fluid pressure chamber is pressurized to a pressure of approximately 5 psi to 750 psi.

4. The pressure flow system as recited in claim 3, wherein the first fluid pressure chamber is pressurized to a pressure of about 5 psi to 200 psi.

5. The pressure flow system as recited in claim 4 wherein the first fluid pressure chamber is pressurized to a pressure of about 5 psi to 100 psi.

6. The pressure flow system as recited in claim 1 wherein the first fluid pressure chamber has a seal to engage bone.

7. The pressure flow system as recited in claim 1 wherein the second fluid pressure chamber has a seal to engage bone.

8. The pressure flow system as recited in claim 1 wherein the second fluid pressure chamber is pressurized to a pressure of about 0 psi to −14.7 psi.

9. A pressure flow system for treating the interior of permeable bone with a fluid which comprises:
a) a fluid pressure chamber having a fluid inlet port and an opening formed through a wall of the chamber sized to allow passage of at least a portion of the bone there through;
b) a seal positioned about the opening conformable to the surface of the bone and capable of fluid-tight engagement therewith; and
c) a source of pressurized fluid wherein when the fluid pressure chamber is pressurized to a pressure greater than atmospheric pressure, fluid from the pressure chamber flows from the pressure chamber through a first end of the interior of the bone and exits the bone outside the pressure chamber; and wherein when the pressure chamber is pressurized to a pressure less than atmospheric pressure, fluid from within the bone flows from the bone through the interior of the bone and exits the bone inside the pressure chamber.

10. A pressure flow system for treating an intact permeable bone with a fluid which comprises:
a) a first fluid pressure chamber having a fluid inlet port and an opening formed through a wall of the chamber sized to allow passage of at least a portion of the bone there through;
b) a seal positioned about the opening conformable to the surface of the bone and capable of fluid-tight engagement therewith;
c) a first source of pressurized fluid having a pressure greater than atmospheric pressure;
d) a second fluid pressure chamber having a fluid inlet port and an opening formed through a wall of the chamber sized to allow passage of at least a portion of the bone therethrough;
e) a seal positioned about the opening conformable to the surface of the bone and capable of fluid-tight engagement therewith; and
f) a second source of pressurized fluid having a pressure less than atmospheric pressure;

11. The pressure flow system as recited in claim 10, wherein when said first pressure source is applied to said first fluid pressure chamber and said second pressure source is applied to said second fluid pressure chamber, fluid flows from said first fluid pressure chamber through the bone and at least partially into said second fluid pressure chamber.

12. The pressure flow system as recited in claim 10, wherein when said first pressure source is applied to said second fluid pressure chamber and said second pressure source is applied to said first fluid pressure chamber, fluid flows through the bone and at least partially into said first fluid pressure chamber.

13. A method for treating an intact bone with a pressure flow system having a fluid which comprises:
Providing;
a) a first fluid pressure chamber having a fluid inlet port and an opening formed through a wall of the chamber sized to allow passage of at least a portion of the bone there through;
b) a seal positioned about the opening conformable to the surface of the bone and capable of fluid-tight engagement therewith;
c) a first source of pressurized fluid having a pressure greater than atmospheric pressure;
d) a second fluid pressure chamber having a fluid inlet port and an opening formed through a wall of the chamber sized to allow passage of at least a portion of the bone there through;
e) a seal positioned about the opening conformable to the surface of the bone and capable of fluid-tight engagement therewith; and
f) a second source of pressurized fluid having a pressure less than atmospheric pressure; placing at least end of bone within one of said first and second chambers; pressuring the first pressure chamber with one of the first and second pressure sources; and pressurizing the second pressure chamber with the other of the first and second pressure sources.

14. The method as recited in claim 13 wherein said first pressure chamber is pressurized with said first pressure source and said second pressure chamber is pressurized with said second pressure source.

15. The method recited in claim 13, wherein said first pressure chamber is pressurized with said second pressure source and said second pressure chamber is pressurized with said first pressure source.

16. The method as recited in claim 13 wherein said first and second pressure chambers are alternately pressurized with said first and second pressure sources.

17. The method as recited in claim 13 wherein said step of placing includes placing a first end of a bone within said first chamber and placing a second end of bone within the second chamber.

18. The method according to claim 13 further comprising the step of forming an opening in said at least one end of the bone.

19. The method as recited in claim 13, wherein said first and second chambers are pressurized to the substantially same pressure greater than atmospheric pressure.

20. The method as recited in claim 13, wherein said first and second chambers are pressurized to substantially the same pressure less than atmospheric pressure.
